(19) United States
(12) Patent Application Publication
(10) Pub. No.: US 2009/0029419 A1
(43) Pub. Date: Jan. 29, 2009

(54) Expression of modified antibodies in avian cells

(75) Inventors: Karen Jervis, Midlothian (GB); Colin Robertson, Midlothian (GB); Joe Conner, Glasgow (GB); William Stimson, Glasgow (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Viragen Incorporated, Plantation, FL (US)

(21) Appl. No.: 10/486,454

(22) PCT Filed: Aug. 9, 2002
(86) PCT No.: PCT/GB02/03692

§ 371 Date: Jul. 12, 2004

(30) Foreign Application Priority Data
Aug. 10, 2001 (GB) 0119497.6

(51) Int. Cl. C12P 21/04; C12N 15/00; C12N 5/06; C12N 15/87; C07K 16/18

(52) U.S. Cl. 435/69.6; 435/320.1; 435/326; 435/455; 530/387.3

(57) ABSTRACT

The present invention relates to construct and methods which allow the expression of immunoglobulins or functional fragments thereof which have been altered to that they are humanised. The expression of the immunoglobulins or fragments thereof takes place in avian cells, and the constructs used have been altered such that the expression levels in avian cells are higher than what would have been expected by simply using a humanised construct. The alterations are based on changing codons so that each amino acid of the codon that is used is the one which is most often found in avians.

The present invention relates to the expression of immunoglobulins, or more specifically, antibodies which have been altered so that the antibody is ‘humanised’, in avian cells. The antibody expression may occur either in vivo or in vitro. In the following document, the terms immunoglobulin and antibody are used interchangeably.

Antibodies are proteins of the immunoglobulin class which are produced on exposure to an antigen. The antibody produced recognises that antigen, binding selectively to it. There are five classes of immunoglobulin and the following text relates primarily to antibodies of the class IgG, although the other classes: IgA, IgD, IgY, IgE and IgM are also included. An antibody molecule is made up of two identical heavy chains linked by disulphide bonds ana two identical light chains. The biological effector functions of an antibody molecule derive from the properties of a constant region, which is identical for antibodies of all specificities within a particular class. It is the variable region that contains the site/s which allows binding to a particular epitope and there is a variable domain at the and of each of the heavy and light chains. These variable domains are followed by a number of constant domains. Thus, the binding of an antibody to an antigen occurs through interactions of the variable domains of each pair of heavy and light chains. Specifically, binding occurs in the areas of the variable regions where there is most variability. These regions are known as hyper variable regions or complementarity determining regions (CDRs).

Generally, with the exception of vaccinations, antibodies are generated from a non-human source such as a mouse. When used in human therapeutic applications, such antibodies are usually recognised as foreign by the immune system. This results in human anti-mouse antibodies being produced, which may reduce the therapeutic effect of the initial antibody or produce undesirable side effects. Techniques have been developed which allow the base of murine antibodies plus those of other species to be manipulated in a way that the original antigen specificity is retained, but all the non-essential parts of the immunoglobulin sequence are replaced with the equivalent human derived sequence. This is known as ‘humanising’ an antibody. By using humanised antibodies, immunogenic responses are largely or highly avoided and effector functions improved.

However, even when an immunoglobulin sequence is humanised there are still problems with the glycosylation pattern of immunoglobulins for use in humans which have not been produced in humans. IgG contains a conserved N-glycosylation site located within the CH2 domain of each heavy chain. The glycosylation pattern of immunoglobulins is highly heterogeneous and has been shown to have a significant effect on the biological, pharmacological and physiochemical properties of the immunoglobulin and include stability of the antibody and half-life, tolerance in patient treatment and interactions with complement components and other Ig receptors. Alterations in the glycosylation pattern of IgG has been linked to symptoms of rheumatoid arthritis. The oligosaccharides are produced in the Golgi apparatus of the cellular interior and is regulated largely by the glycosyltranaferases present in this organelle. Currently, antibodies for therapeutic applications are being produced in a variety of cell lines and transgenically, but many of these systems are not particularly suitable in terms of glycosylation. The same antibody produced in different cell lines and animals may therefore be afforded different characteristics which may result in differing functions and pharmacokinetics. The expression of glycosyltranaferases differs with different cell types, with the result that the glycosylation pattern of the protein produced differs from that produced by other cell types. In particular, it has been noted that non-human mammalian cell lines, for example hamster cell lines, show markedly different glycosylation patterns to humans and therefore are likely to cause problems with immunogenicity. It is known that normal Chinese Hamster Ovary (CHO) Cells, which are the standard used in the industry for the manufacture of recombinant proteins, do not express the enzyme N-acetylglucosaminyltransferase-III (GlcNAcT-III), which has the role of synthesising carbohydrates which contain GlcNAc (Campbell & Stanley, 1984). Since GlcNAc is expressed in human immunoglobulin G, it is known that human proteins expressed in CHO cells are not glycosylated in this manner. It is also interesting to note that chicken IgG's also possess this oligosaccharide. It has been found by experts in the area, such as that reported by Raju et al. 2002, that the glycosylation patterns of avian cells are far more similar than non-human mammalian glycosylation patterns to human glycosylation patterns. It therefore can be seen that it would be highly advantageous to be able to utilise chicken cells as a mode of production for humanised recombinant proteins, compared to production of such proteins in the standard mammalian cells.

U.S. Pat. No. 5,225,539 entitled “Recombinant Altered Antibodies and Method of Making Altered Antibodies” describes a method of replacing the complementarity determining regions of the heavy or light chain variable domains of the receiving antibody with the corresponding complementarity determining regions of a different antibody with a different specificity. This method, known as ‘CDR grafting’ is not the only method to produce humanised antibodies, but is the most well known to those knowledgeable in the field. The present invention relates to antibodies which have been manipulated in any manner which has the result of producing an antibody which is more human-like in sequence than the wild-type sequence.

However, although U.S. Pat. No. 5,225,539 describes a general method which allows altered antibodies to be produced, the methods described are directed towards production of the altered antibodies in mammalian cells and does not envisage the problems and advantages that arise when producing antibodies in avian cells using this technology.

While the methodology described in Winters' U.S. Pat. No. 5,225,539 is extremely useful for the production of CDR grafted antibodies in mammalian cells, it can be seen that it would also be useful to be able to easily produce such CDR grafted and humanised antibodies in avian calls. The production of such antibodies in avian cells would have a significant advantage over the production in mammalian cells if they are to be used as a human therapeutic. This is because the glycosylation pattern of human antibodies is more similar to avian antibodies than the glycosylation of mammalian antibodies. As previously mentioned, it is known that the glycosylation pattern can have a significant effect on the bioactivity, immunogenicity and therefore tolerance to the treatment and also the pharmacokinetics of the antibody itself, and therefore it would be extremely useful to produce antibodies for human use of which the glycosylation pattern is close to that of human antibodies.

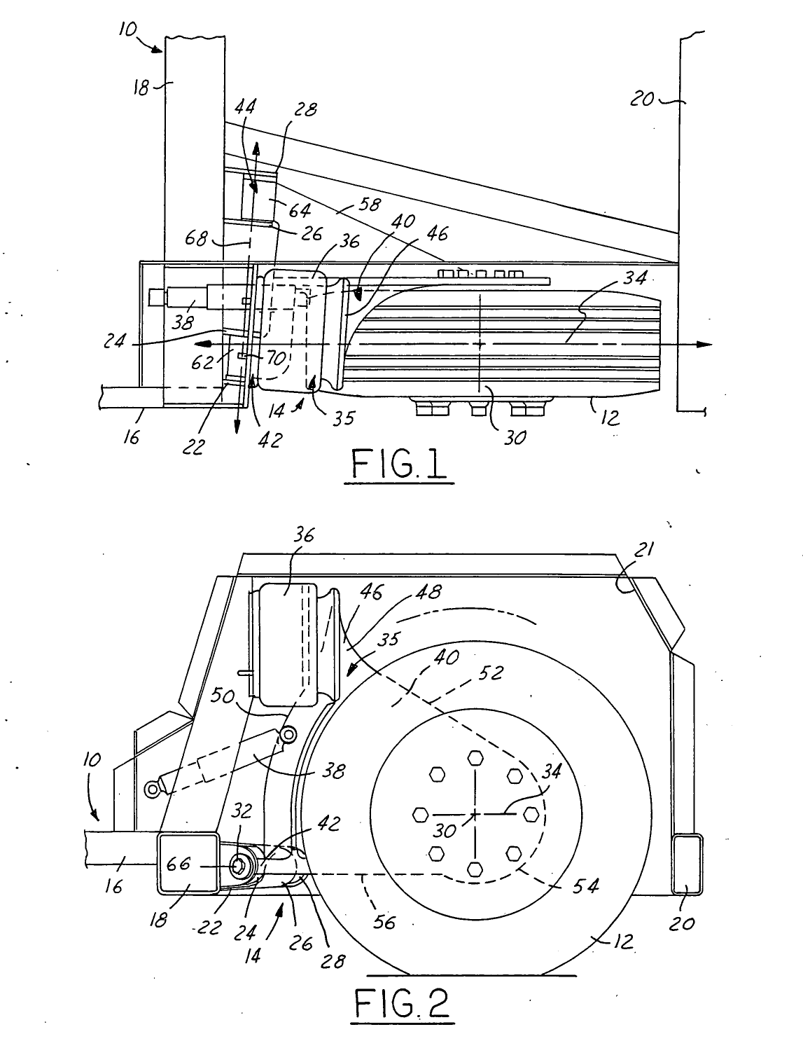

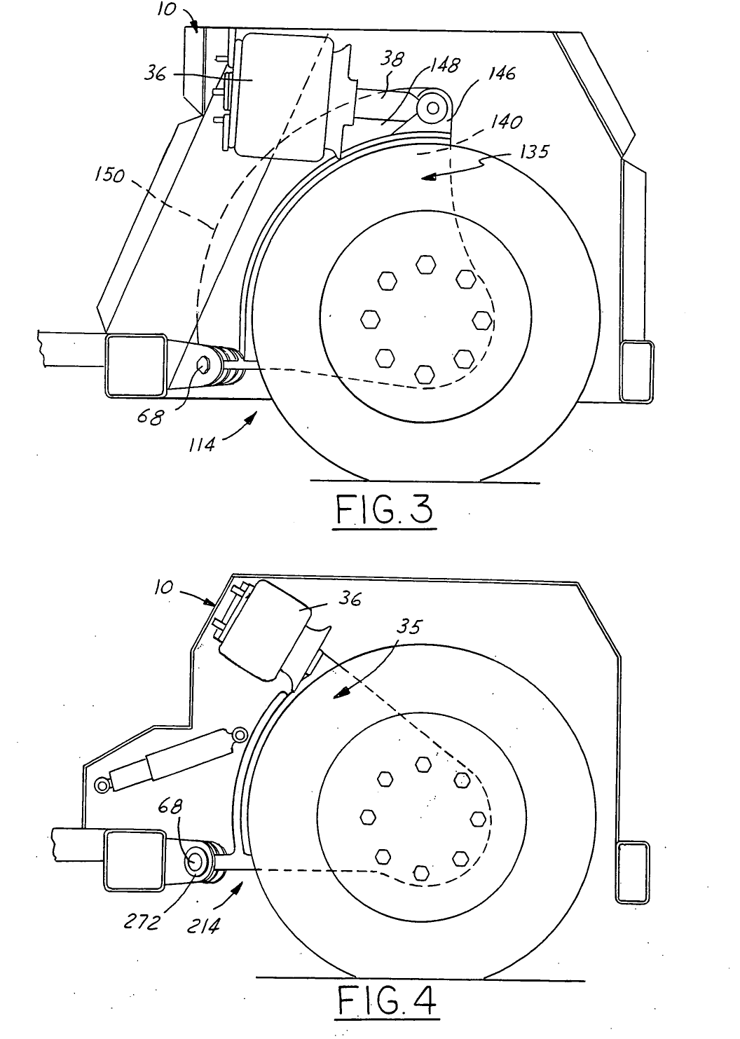

It is also known that the yield of proteins produced in non-avian transgenic animals, can be limited. It is common practice to have to alter the culture conditions or use expression vectors in order to obtain commercially viable expression levels. The inventors have shown that the expression of such proteins, especially immunoglobulins and fragments thereof, in avian cells is in general, higher than that observed in non-avian animals. It can therefore be seen that it would be useful and more time and cost-effective to produce antibodies for human therapeutic use in avian cells. FIG. 1 shows the result of a Western blot to compare the expression levels of the R24 protein in Chicken Hepatocellular (LMH) cells, as compared to expression in Chinese Hamster Ovary (CHO) cells. FIG. 2 illustrates a comparison between R24 protein produced in LMH and CHO cells, analysed by human IgGl ELISA. It can be seen, that there is a significantly higher level of protein produced in the LMH cells as compared to the CHO cells. The inventors believe that this is due to certain differences between the translational machinery of the cell types in that the LMH cells are more efficient in post-translationally modifying the protein, than mammalian cells. This belief is emphasised when the RNA message produced by both cell types is analysed by PCR gel and the levels produced by both types are similar.

One of the major problems of producing modified antibodies in avian cells is that the codon usage in avians differs from that in humans. Therefore, the methodology described in U.S. Pat. No. 5,225,539 is not sufficient to allow the making of altered antibodies in avian cells efficiently.

There is also the problem of actually producing that which is coded for in a genetic construct in avian cells. U.S. Pat. No. 4,816,397 describes methods of producing multi-chain polypeptides or proteins generally, however again the methods are discussed mainly with regard to mammalian cells and do not address the problems which occur when producing multi-chain polypeptides, such as antibodies, in avian cells. It should be noted that there are a number of Patents and Patent Applications which address the problem of producing basic single chain proteins in avian cells, however in these cases they do not refer to the production of complex multi-chain polypeptides, such as antibodies, which pose their own problems.

It can therefore be seen that it would be beneficial to provide a method of producing and expressing modified antibodies which have similar glycosylation patterns to those naturally produced in humans or more similar than the glycosylation patterns obtained from antibodies from culture in mammalian cells.

It can also be seen that it would be beneficial to provide a method of producing and expressing modified antibodies in avian cells, as specifically producing and expressing humanised antibodies in avian cells, so that they can be used as human therapeutics.

It would also be extremely useful if the expression of the modified antibodies could be specifically in the egg of a genetically modified avian, so that the antibody can easily be collected and purified.

It is therefore a first object of the present invention to provide antibodies which have a glycosylation pattern which is more similar to naturally occurring human glycosylation patterns than those antibodies produced in mammalian cells.

It is a further object of the present invention to provide a method of expressing such humanised antibodies in avian cells in vitro and in vivo.

It is a further object of the present invention to provide a method of expressing humanised antibodies in avian cells which are known to produce higher yields than observed in non-avian cell systems.

It is a further object of the present invention to provide a method of expressing humanised antibodies in avian cells in vitro and in vivo.

It is a yet further object of the present invention to provide antibodies for therapeutic use, which have glycosylation patterns which are more similar to those naturally occurring in humans, than the glycosylation patterns observed in antibodies produced in mammalian cells.

A still further object of the present invention is to provide a construct which can be delivered into avian cells, which will allow the production of antibodies or humanised antibodies.

A final object of the present invention is to provide a method of expressing humanised antibodies in avian cells, so that they are specifically produced in the egg white or egg yolk of an avian.

According to a first aspect of the present invention, there is provided a DNA construct which when transfected into an avian cell will allow the production of an antibody molecule or functional fragment of said molecule, and which comprises at least one sequence which comprises the variable domain of an immunoglobulin heavy chain and at least one sequence which comprises the variable domain of an immunoglobulin light chain, and wherein the DNA construct is based on a non-avian sequence and one or more of the codons in the DNA construct have been altered such that for the amino acid being encoded, the codon used is that which most frequently appears in avians.

Preferably, the construct also contains an avian signal peptide sequence.

Preferably the construct is cloned into a vital vector such as a lentivirus vector

Most preferably, the avian signal peptide sequence is a signal peptide sequence from an egg white protein such as lysozyme, ovalbumin, ovatransferrin or ovamucoid.

Most preferably, the construct also includes immunoglobulin constant regions for dimerisation and recruitment of effector functions.

Most preferably, the immunoglobulin constant regions are CH2 and CH3 of any IgG class.

Still more preferably, the immunoglobulin constant regions are human constant regions in order to provide a humanised antibody.

Preferably, the construct may be transfected into an avian cell using lipofection.

Alternatively, the construct is transfected into an avian cell using electroporation.

A further option is that the construct may be directly injected into the nucleus of an avian, into the germinal disc of an oocyte.

Preferably, codon usage in the construct is maximised for those codons most frequently appearing in avians. That is, each codon is altered so that it still codes for the same amino acid, but uses the codon most often found to code for that amino acid in avians.

According to a second aspect of the present invention, there is provided an avian cell, containing the construct of the first aspect, which expresses an immunoglobulin molecule or functional fragment of said molecule.

Preferably the expressed immunoglobulin molecule or fragment thereof shows an avian glycosylation pattern.

Preferably the immunoglobulin or fragment thereof is expressed at a higher expression level than a standard human construct or humanised construct.

According to a third aspect of the present invention, there is provided a method for producing avian cells capable of expressing an immunoglobulin molecule or functional fragment of said molecule, comprising transfecting an avian cell with the DNA construct of the first aspect.

Preferably the avian cell is a chicken cell, but may also be duck, turkey, quail, or ostrich.

According to a fourth aspect of the present invention there is provided an immunoglobulin or functional fragment thereof produced using the method of the third aspect.

According to a fifth aspect of the present invention there is provided a transgenic avian, which expresses the construct of the first aspect.

Preferably the antibody molecule, or functional fragment of said molecule, that is coded for by the construct is expressed in an egg of the transgenic avian.

Most preferably the construct is expressed in the egg white.

Alternatively the construct is expressed in the egg yolk.

Preferably the immunoglobulin shows an avian glycosylation pattern.

The present invention will now be illustrated, by way of example only, with reference to the following Figures in which:

FIG. 1 shows a Western blot showing the differences in protein expression between chicken and mammalian cells; and

FIG. 2. is a graph illustrating the differences between protein expression levels in chicken and mammalian cells, as analysed by ELISA; and

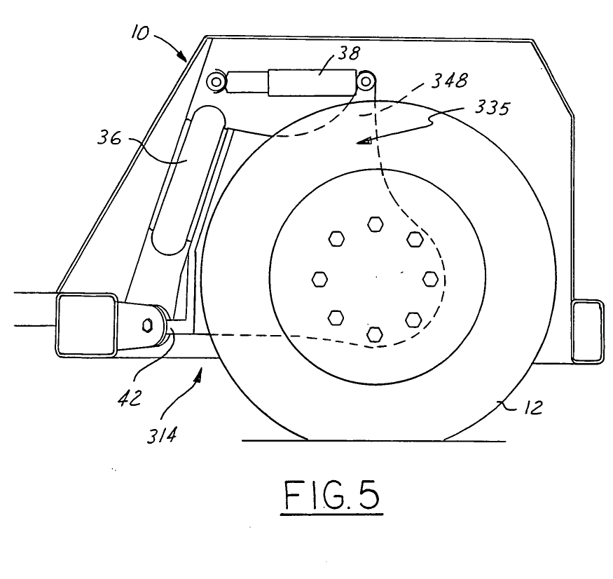

FIG. 3 is a table giving the frequency of codon usage in chickens.

And with reference to the following sequences in which;

SEQUENCE ID 1 is the sequence of human IgG Fc used for construction of chimeric and humanised minibodies; and

SEQUENCE ID 2 is the sequence of the chickenised IgG Fc DNA sequence.

SEQUENCE ID 3 is the nucleotide alignment of the original and chickenised human IgG Fc.

SEQUENCE ID 4 is the amino acid alignment of original and chickenised human IgG Fc.

SEQUENCE ID 5 shows the chickenised R24 nucleotide sequence.

SEQUENCE ID 6 shows the complete chickenised nucleotide sequence of the R24 chimeric minibody

Generating a Construct

In this example a construct is produced, which allows a humanised murine R24 antibody to be produced in chickens.

The DMA encoding the single chain variable fragment is designed in silico so that it can then be directly synthesised using standard methods.

The starting sequences are human Vh and Vl sequences which may be obtained from human IgM antibody. Vh and Vl complementarity determining regions (CDRs) of another immunoglobulin (which in this case is the murine R24 immunoglobulin) are identified by standard methods (e.g. see Antibodies-Structure and Sequence at http://www.bioinf.org.uk/abs) and the R24 CDRs are swapped directly into the human immunoglobulin framework.

The 3′ end of the Vh DNA sequence is linked to the 5′ end of the Vl DNA sequence by a (Gly₄Ser)₃ peptide linker, as seen in SEQUENCE ID 1. Included at the 3′ end of the Vl sequence is a sequence encoding a Bam HI restriction site. This gives the humanised R24 sequence 1. An IgG1 leader sequence is linked to the 5′ end of Vh with the inclusion of an Eco RI restriction site.

To provide the constant region of the immunoglobulin 2, human IgG1 CH2/CH3 (Fc) DNA is then cloned by RT-PCR from RNA. The primers incorporate Bam HI and Sal I restriction sites and can be seen in SEQUENCE ID 2. The amplified DNA fragment is cloned directly following PCR using the PCR cloning vector pGEM-T (Promega). E coli DH5α cells are transformed with the ligated plasmid, plated out on amp selection media and colonies screened the following day by PCR with M13 vector primers. Positive clones with the appropriately sized insert can then be selected and plasmid DNA can be prepared and inserts sequenced to confirm the presence of immunoglobulin constant region DNA. The insert from one positive DNA clone is removed by Bam HI/Sal I digestion and ligated into pGEM 3Z (Promega), digested with the same restriction enzymes. After transformation and overnight growth on amp media, colonies are screened by PCR with M13 vector primers and plasmid prepared from one positive clone.

One μg of the R24 gene synthesis product 1 is digested with EcoRI/BamHI and ligated into plasmid hFc digested with the same enzymes. After transformation and overnight growth on amp media, colonies are screened by PCR with M13 vector primers and plasmids prepared from clones with the appropriately sized insert.

The entire insert DNA is then removed from pGEM3z by digestion with the restriction enzymes EcoRI/Sal1 and ligated into the mammalian expression vector pCIneo (Promega) digested with the same enzymes. After transformation and overnight growth on amp media, colonies are screened by restriction digestion (EcoRI/SalI) of plasmid preparations. Plasmids may be sequenced to confirm the presence of the minibody genes.

The insert can then be used to form a construct for insertion into an avian cell. The insert, comprising the Vh/Vl CDRs transplanted into a human immunoglobulin framework 1 along with immunoglobulin constant domains 2 is removed from the pCIneo vector by BgIII/SfiI digestion. The fragment that is gained by this digestion consists of:
a promoter/enhancer 6,
an intron 4,
the minibody which comprises the R24 variable regions 1 and the CH2 and CH3 constant regions 2, and
a poly A tail 3

In order for the construct to be suitable for expression in an avian cell, the immunoglobulin leader sequence is exchanged for an avian specific sequence such as the lysozyme signal peptide sequence 5. Also, both the R24 variable section coding sequence 1 and the CH2/CH3 constant region coding sequence 2 are chickenised.

‘Chickenising’ a Construct

Chickenising is defined as the alteration of codon usage such that it is maximised for those codons most frequently used in chickens. For expression in transgenic chickens the codons of constructs are optimised for most frequent codon usage in chickens. However, it can be seen that the optimisation could be for the most frequent codon usage of any avian species.

### EXAMPLE

Chickenising the Human IgG Fc DNA Sequence

For expression in transgenic chickens the codons of the chimaeric and humanised minibody versions of R24 are optimised for most frequent codon usage in chickens (Gallus gallus). A table detailing frequency of codon usage was downloaded from http://www.kazusa.or.jp and is reproduced in FIG. 3.

For an example of how chickenisation is carried out, it can be seen that the amino acid Valine is encoded by 4 different codons, GTG, GTA, GTT and GTC with GTG used most frequently in chickens (46% GTG, 11% GTA, 19% GTT and 23% GTC*). To chickenise the human IgG Fc DNA, all valine codons were converted to GTG. Lysine is encoded by two different codons, AAG and AAA, with AAG used most frequently in chickens (58% vs 42%). All AAA codons in the sequence were converted to AAG. Not all codons required alteration. For example, the two codons for aspartic acid, GAT and GAC are used with almost equally (48% vs 52%) and are not changed during the chickenisation. * Figures as given in FIG. 3=99%

Sequence ID 1 shows the codons for the original human IgG Fc DNA sequence. Sequence ID 2 shows the chickenised version of this. Sequence ID 3 shows an alignment of the nucleotide sequences, a dot indicates a sequence match and the missing dots show where the codons have been altered. Sequence ID 4 is an alignment of the amino acid sequences which show that despite the alterations to various codons the amino acids are still 100% identical. Sequence ID 5 and 6 show the chickenised R24 scFv sequence and complete chickenised R24 minibody respectively.

Inserting the Construct Into an Avian Cell

There are a number of possible, methods that can be used for transfection of an avian cell with the construct. Transfection can either be transient or stable.

In transient transfection, supercoiled plasmid containing the gene of interest is introduced into the nucleus of the target cells at high copy number for short periods of time (usually 24-96 hrs). During transient transfections the DNA does not integrate into the cellular chromosomes.

For stable transfection either linear or plasmid DNA can be introduced into the target cells and will either integrate into the chromosomes or be maintained as a stable episome. Linear DNA is optimal for stable integration but is taken up less efficiently than supercoiled plasmid. Cells in which the DNA has integrated or is maintained as a stable episome can be distinguished by selectable markers. For transfections with pCIneo, the plasmid carries the neomycin phosphotransferase gene which confers resistance to aminoglycosides such as G418. Culturing cells in the presence of G418 selects for those that carry the integrated or episomal DNA.

A variety of methods are available for the introduction of DNA into mammalian cells and these include calcium phosphate coprecipitation (Graham, R. L. and van der Erb, A J A (1973) Virology 52, 456.) and electroporation (Andreason, G. L. and Evans, G. A. (1988) BioTechniques 6, 650; Shigikawa, K. and Dover, W. J., (1988) BioTechniques 6, 742) but these have largely been superseded by cationic liposome-mediated transfection. (Felgner, J. et al (1993) J Tiss Cult Metho. 15, 63). Other compounds known to mediate transfection of mammalian cells include lipopolyamines (Remy, J-S, Sirlin, C., Vierling, P and Behr, J-P. (1994) Bioconjugate Chem. 5, 647) and dendrimers (Haensler, J. and Szoka, F C (Jr) (1993) Bioconjugate Chem 4, 372.).

Cationic liposomes, lipolyamines and dendrimers coat the DNA to be transfected and mediate its passage through the cell membrane. A variety of factors influence the efficiency of transfection and these include cell type, media type and presence of serum and antibiotics, amount and quality of plasmid DNA and cytotoxicity of transfection reagent. All of these usually have to be optimised for each cell type and plasmid construct.

Alternatively, zygotic injection of the construct could also be used to incorporate the construct.

Alternatively the construct may be cloned into a viral vector such as a lentivirus vector and such vectors are commercially available. Lentiviruses as vectors have been developed from slow retroviruses, such as equine infectious anaemia virus (EIAV), feline immunodeficiency virus (FIV) or Human Immunodeficiency Virus (HIV). The significant advantage using a lentiviral vector is that the virus will infect cells that are not dividing, which is appropriate to certain cell types of the present invention.

If the construct contains a promoter such as an egg white protein signal peptide, transgenic avians can then be produced which lay eggs with the antibody of interest in the egg white.

The chickenised construct may also be designed for insertion into a gene contained within a plasmid, for example it may be designed for insertion into a lysozyme gene contained within a plasmid. The ATG site on the lysozyme gene in the plasmid is destroyed by creating a SalI site so that the lysozyme protein is not expressed. The chickenised construct, which has its own ATG can then be cloned into the SalI site.

Various modifications may bs made to the invention herein described, without departing from the scope thereof. For example, any appropriate immunoglobulin sequence may be used and any appropriate avian species may be used in place of chickens with the codon bias changing appropriately.

1. A DNA construct which when transfected into an avian cell will allow the production of an antibody molecule or functional fragment of said molecule, and which comprises at least one sequence comprising the variable domain of an immunoglobulin heavy chain and at least one sequence comprising the variable domain of an immunoglobulin light chain, and wherein the DNA construct is based on a non-avian sequence and one or more of the codons in the DNA construct have been altered such that for the amino acid being encoded, the codon used is that which most frequently appears in avians.

2. A DNA construct as claimed in claim 1, wherein the construct also contains an avian signal peptide sequence.

3. A construct as claimed in the previous claims, wherein the construct is cloned into a viral vector.

4. A construct as described in claim 3, wherein the viral vector is a lentivirus vector.

5. A DNA construct as claimed in claims 2 to 4, wherein the avian signal peptide sequence is a signal peptide sequence from an egg white protein.

6. A DNA construct as described in claim 5, wherein the egg white protein is chosen from the list lysozyme, ovalbumin, ovatransferrin or ovamucoid.

7. A DNA construct as claimed in any of the previous claims, wherein the construct also includes immunoglobulin constant regions for dimerisation and recruitment of effector functions.

8. A DNA construct as claimed in claim 7, wherein the immunoglobulin constant regions are CH2 and CH3.

9. A DNA construct as claimed in claims 7 or 8, wherein the immunoglobulin constant regions are human constant regions.

10. A DNA construct as described in any of the previous claims, wherein the construct may be transfected into an avian cell using electroporation.

11. A DNA construct as claimed in claims 1 to 9, wherein the construct may be transfected into an avian cell using lipofection.

12. A DNA construct as claimed in claim 1 to 9, wherein the construct may be directly injected into the nucleus of an avian.

13. A DNA construct as claimed in claim 12, wherein the construct may be directly injected into the germinal disc of an oocyte.

14. A DNA construct as described in any of the previous claims, wherein codon usage in the construct is maximised for those codons most frequently appearing in avians.

15. An avian cell containing the construct described in claims 1 to 15, which expresses an immunoglobulin molecule or functional fragment of said molecule.

16. An avian cell as described in claim 16, wherein the expressed immunoglobulin molecule or functional fragment thereof shows an avian glycosylation pattern.

17. An avian cell as described in claims 16 or 17, wherein the immunoglobulin or fragment thereof is expressed at a higher expression level than a standard human construct or humanised construct.

18. A method for producing avian cells capable of expressing an immunoglobulin molecule or functional fragment of said molecule, comprising transfecting an avian cell with the DNA construct as described in claims 1 to 15.

19. Preferably the avian cell is selected from a list of chicken cell, duck cell, turkey cell, quail cell or ostrich cell.

20. An immunoglobulin or functional fragment thereof which is produced using the method described in claims 19 and 20.

21. A transgenic avian which expresses the construct described in claims 1 to 15.

22. A transgenic avian as described in claim 22, wherein the molecule coded for by the construct is expressed in the egg of the transgenic avian.

23. A transgenic avian as claimed in claim 23, wherein the construct is expressed in the egg white.

24. A transgenic avian as described in claim 23, wherein the construct is expressed in the egg yolk.

25. A transgenic avian as described in claims 22 to 25, wherein the expressed immunoglobulin shows an avian glycosylation pattern.


SEQUENCE LISTING (6 sequences)

SEQ ID NO: 1 - DNA, 733 bp, Homo sapiens
accttgcaggatccgcaagacccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgataagtcgacacgtgatc

SEQ ID NO: 2 - DNA, 733 bp, Gallus gallus
accttgcaggatccgccagacccaagtcctgcgacaagacccacacatgcccaccctgcccagcccccgagctgctggggggaccctccgtgttcctgttccccccaaagcccaaggacaccctgatgatctcccgcacccccgaggtgacatgcgtggtggtggacgtgagccacgaggaccccgaggtgaagttcaactggtacgtggacggcgtggaggtgcacaacgccaagacaaagccccgcgaggagcagtacaacagcacctaccgcgtggtgagcgtgctgaccgtgctgcaccaggactggctgaacggcaaggagtacaagtgcaaggtgtccaacaaggccctgccagcccccatcgagaagaccatctccaaggccaaggggcagccccgcgagccacaggtgtacaccctgcccccatcccgcgaggagatgaccaagaaccaggtgagcctgacctgcctggtgaagggcttctaccccagcgacatcgccgtggagtgggagagcaacgggcagcccgagaacaactacaagaccaccccccccgtgctggactccgacggctccttcttcctgtacagcaagctgaccgtggacaagagcaggtggcagcaggggaacgtgttctcctgctccgtgatgcacgaggccctgcacaaccactacacccagaagagcctctccctgtcccccggcaagtgataagtggacacctgatc

SEQ ID NO: 3 - protein, 242 aa, Homo sapiens
  MISC_FEATURE (238)..(239): Xaa can be any naturally occurring amino acid
LAGSARPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKXXVDT

SEQ ID NO: 4 - protein, 242 aa, Gallus gallus; identical to SEQ ID NO: 3
  MISC_FEATURE (238)..(239): Xaa can be any naturally occurring amino acid

SEQ ID NO: 5 - DNA, 814 bp, Gallus gallus
  misc_feature (13)..(66): lysozyme_leader
ggccgggtcgacatgaggtctttgctaatcttggtgctttgcttcctgcccctggctgctctgggggatgtgcagctggtggagtccgggggaggcctggtgcagcccggagggtcccgcaagctctcctgcgccgcctccggattcaccttcagcaacttcggaatgcactgggtgcgccaggcccccgagaaggggctggagtgggtgggatacatcagcagcggcggcagctccatcaactacgccgacaccgtgaagggccgcttcaccatctccagagacaaccccaagaacaccctgttcctgcagatgaccagcctgaggtccgaggacacagccatctactactgcaccagagggggaaccgggaccagatccctgtactacttcgactactggggccagggcgccacactgatcgtgtcctccgggggaggcggctccgggggaggcggctccgggggaggcggctccgatatccagatgacacagatcacaatcggcaattttctgaactggtaccagcaggaaccagatggatctctgaagctgctgatctactacacatctagactgcagtccggagtgccatccaggttcagcggctgggggtctggaacagattactctctgaccattagcaacctggaggaagaggatatctcctccctgtctgtgtctctgggagacagagtgatcatcagctgcagggctagccaggacgccaccttcttctgccagcagggcaagacactgccctacaccttcggaggggggaccaagctggagatcaagcgcggatccgccgccg

SEQ ID NO: 6 - DNA, 1526 bp, Gallus gallus
ggccgggtcgacatgaggtctttgctaatcttggtgctttgcttcctgcccctggctgctctgggggatgtgcagctggtggagtccgggggaggcctggtgcagcccggagggtcccgcaagctctcctgcgccgcctccggattcaccttcagcaacttcggaatgcactgggtgcgccaggcccccgagaaggggctggagtgggtgggatacatcagcagcggcggcagctccatcaactacgccgacaccgtgaagggccgcttcaccatctccagagacaaccccaagaacaccctgttcctgcagatgaccagcctgaggtccgaggacacagccatctactactgcaccagagggggaaccgggaccagatccctgtactacttcgactactggggccagggcgccacactgatcgtgtcctccgggggaggcggctccgggggaggcggctccgggggaggcggctccgatatccagatgacacagatcacatcctccctgtctgtgtctctgggagacagagtgatcatcagctgcagggctagccaggacatcggcaattttctgaactggtaccagcaggaaccagatggatctctgaagctgctgatctactacacatctagactgcagtccggagtgccatccaggttcagcggctgggggtctggaacagattactctctgaccattagcaacctggaggaagaggatatcgccaccttcttctgccagcagggcaagacactgccctacaccttcggaggggggaccaagctggagatcaagcgcggatccgccagacccaagtcctgcgacaagacccacacatgcccaccctgcccagcccccgagctgctggggggaccctccgtgttcctgttccccccaaagcccaaggacaccctgatgatctcccgcacccccgaggtgacatgcgtggtggtggacgtgagccacgaggaccccgaggtgaagttcaactggtacgtggacggcgtggaggtgcacaacgccaagacaaagccccgcgaggagcagtacaacagcacctaccgcgtggtgagcgtgctgaccgtgctgcaccaggactggctgaacggcaaggagtacaagtgcaaggtgtccaacaaggccctgccagcccccatcgagaagaccatctccaaggccaaggggcagccccgcgagccacaggtgtacaccctgcccccatcccgcgaggagatgaccaagaaccaggtgagcctgacctgcctggtgaagggcttctaccccagcgacatcgccgtggagtgggagagcaacgggcagcccgagaacaactacaagaccaccccccccgtgctggactccgacggctccttcttcctgtacagcaagctgaccgtggacaagagcaggtggcagcaggggaacgtgttctcctgctccgtgatgcacgaggccctgcacaaccactacacccagaagagcctctccctgtcccccggcaagtgataagtcgacacctgatc